(19) United States
(12) Patent Application Publication
(10) Pub. No.: US 2024/0239872 A1
(43) Pub. Date: Jul. 18, 2024

(54) COMBINATION OF PVY MONOCLONAL ANTIBODIES CAPABLE OF RECOGNIZING DIFFERENT ANTIGENIC DETERMINANTS, AND USE THEREOF

(71) Applicant: Shandong Agricultural University, Tai'an (CN)

(72) Inventors: Xiangdong LI, Tai'an (CN); Zhiyong Yan, Tai'an (CN); Cuiling Zhao, Tai'an (CN); Qing Zhu, Tai'an (CN); Xiuqi Mu, Tai'an (CN); Yanping Tian, Tai'an (CN); Chao Geng, Tai'an (CN)

(21) Appl. No.: 18/384,035

(22) Filed: Oct. 26, 2023

(30) Foreign Application Priority Data
Jan. 17, 2023 (CN) 202310062606.7

(51) Int. Cl. C07K 16/10; G01N 33/569

(52) U.S. Cl. CPC C07K 16/10; G01N 33/56983; C07K 2317/565

(57) ABSTRACT

The invention discloses a combination of PVY monoclonal antibodies capable of recognizing different antigenic determinants, and a use thereof, and belongs to the biotechnological field. Monoclonal antibodies N1, M1, M2, M3 and C1 obtained in the invention have specific CDRs, and are significantly different from existing reported monoclonal antibodies. The five monoclonal antibodies and a combination thereof can recognize all or most PVY isolates which have been reported, thus reducing the possibility of detection omissions; and the five monoclonal antibodies do not react with other congeneric viruses and non-congeneric viruses, thus reducing the possibility of detection errors. Therefore, the monoclonal antibodies and the combination thereof can realize accurate PVY detection.

### REFERENCE TO AN ELECTRONIC SEQUENCE LISTING

The contents of the electronic sequence listing (SPD20230012US_SEQ; Size: 58 K bytes; and Date of Creation: Oct. 26, 2023) is herein incorporated by reference in its entirety. The contents of the electronic sequence listing in no way introduces new matter into the specification.

### BACKGROUND OF THE INVENTION

### 1. Technical Field

The invention relates to the biotechnological field, in particular to a combination of PVY monoclonal antibodies capable of recognizing different antigenic determinants, and a use thereof.

### 2. Description of Related Art

Potato virus Y (PVY), as a representative species of potyvirus, typically infects solanaceous crops such as potato, tobacco and chilli, and severely compromises the yield and quality of crops. PVY may cause mosaic disease, vein necrosis, stalk dwarfing, tuber necrosis, ringspots, and other symptoms of potato, and mottles, mosaic disease, vein necrosis and other symptoms of tobacco, and even can result in a crop loss of 80% in severe cases. PVY may spread through aphids in a non-persistent manner, or through juice contact, and may spread through potato seeds. Timely and accurate identification of PVY makes it possible to realize the occurrence of PVY and provides a theoretical basis for monitoring, warning and comprehensive control of PVY.

Detection omissions or errors may lead to incorrect detection results, are disadvantageous to effective control of PVY, and severely impair safety production of crops. Commercial PVY monoclonal antibodies Mab1128, Mab1129 and Mab1130 can recognize antigenic determinants ²⁵NLNKEK³⁰, ¹⁶RPEQGSIQSNP²⁶ and ^{5I}DAGGS¹⁰ of coat protein (CP). However, many PVY isolates do not include these three antigenic determinants, so detection omissions will be caused when these three monoclonal antibodies are used for PVY detection. PVY polyclonal antibodies may undergo a cross reaction with other viruses of potyvirus, so detection errors will be caused when polyclonal antibodies are used for PVY detection.

It is the key to safety protection of important crops such as potato and chilli is to screen out PVY monoclonal antibodies capable of recognizing new antigenic determinants, specify the conservativeness and specificity of antigenic determinants, and combine the PVY monoclonal antibodies to realize accurate virus detection to prevent detection errors and omissions.

### BRIEF SUMMARY OF THE INVENTION

In view of the prior art, the objective of the invention is to provide a combination of monoclonal antibodies capable of detecting all or most PVY isolates, and a use thereof.

To fulfill the above objective, the invention adopts the following technical solution:

In a first aspect, the invention provides a monoclonal antibody for detecting PVY, wherein the monoclonal antibody is at least one of monoclonal antibodies N1, M1, M2, M3 and C1;

The monoclonal antibody N1 comprises VHCDR1, VHCDR2 and VHCDR3 having amino acid sequences as set forth in SEQ ID NO:1-3, as well as VLCDR1, VLCDR2 and VLCDR3 having amino acid sequences as set forth in SEQ ID NO:4-6;

The monoclonal antibody M1 comprises VHCDR1, VHCDR2 and VHCDR3 having amino acid sequences as set forth in SEQ ID NO:7-9, as well as VLCDR1, VLCDR2 and VLCDR3 having amino acid sequences as set forth in SEQ ID NO:10-12;

The monoclonal antibody M2 comprises VHCDR1, VHCDR2 and VHCDR3 having amino acid sequences as set forth in SEQ ID NO:13-15, as well as VLCDR1, VLCDR2 and VLCDR3 having amino acid sequences as set forth in SEQ ID NO:16-18;

The monoclonal antibody M3 comprises VHCDR1, VHCDR2 and VHCDR3 having amino acid sequences as set forth in SEQ ID NO:19-21, as well as VLCDR1, VLCDR2 and VLCDR3 having amino acid sequences as set forth in SEQ ID NO:22-24;

The monoclonal antibody M2 comprises VHCDR1, VHCDR2 and VHCDR3 having amino acid sequences as set forth in SEQ ID NO:25-27, as well as VLCDR1, VLCDR2 and VLCDR3 having amino acid sequences as set forth in SEQ ID NO:28-30.

Further, a heavy-chain variable region of the monoclonal antibody N1 has an amino acid sequence as set forth in SEQ ID NO:31, and a light-chain variable region of the monoclonal antibody N1 has an amino acid sequence as set forth in SEQ ID NO:32; a heavy-chain variable region of the monoclonal antibody M1 has an amino acid sequence as set forth in SEQ ID NO:33, and a light-chain variable region of the monoclonal antibody M1 has an amino acid sequence as set forth in SEQ ID NO:34; a heavy-chain variable region of the monoclonal antibody M2 has an amino acid sequence as set forth in SEQ ID NO:35, and a light-chain variable region of the monoclonal antibody M2 has an amino acid sequence as set forth in SEQ ID NO:36; a heavy-chain variable region of the monoclonal antibody M3 has an amino acid sequence as set forth in SEQ ID NO:37, and a light-chain variable region of the monoclonal antibody M3 has an amino acid sequence as set forth in SEQ ID NO:38; a heavy-chain variable region of the monoclonal antibody C1 has an amino acid sequence as is set forth in SEQ ID NO:39, and a light-chain variable region of the monoclonal antibody C1 has an amino acid sequence as set forth in SEQ ID NO:40.

Further, a heavy chain of the monoclonal antibody N1 has an amino acid sequence as set forth in SEQ ID NO:41, and a light chain of the monoclonal antibody N1 has an amino acid sequence as set forth in SEQ ID NO:42; a heavy chain of the monoclonal antibody M1 has an amino acid sequence as set forth in SEQ ID NO:44, and a light chain of the monoclonal antibody M1 has an amino acid sequence as set forth in SEQ ID NO:44; a heavy chain of the monoclonal antibody M2 has an amino acid sequence as set forth in SEQ ID NO:45, and a light chain of the monoclonal antibody M2 has an amino acid sequence as set forth in SEQ ID NO:46; a heavy chain of the monoclonal antibody M3 has an amino acid sequence as set forth in SEQ ID NO:47, and a light chain of the monoclonal antibody M3 has an amino acid sequence as set forth in SEQ ID NO:48; a heavy chain of the monoclonal antibody C1 has an amino acid sequence as set forth in SEQ ID NO:49, and a light chain of the monoclonal antibody C1 has an amino acid sequence as set forth in SEQ ID NO:50.

Preferably, the monoclonal antibody is any one of combinations of monoclonal antibodies (1)-(3):

(1) a combination of the monoclonal antibody M1 and the monoclonal antibody M2;

(2) a combination of the monoclonal antibody M1 and the monoclonal antibody C1;

(3) a combination of the monoclonal antibody M2 and the monoclonal antibody C1.

In a second aspect, the invention provides a coding gene of a monoclonal antibody, comprising:

nucleotide sequences respectively coding a heavy chain and a light chain of a monoclonal antibody N1, as set forth in SEQ ID NO:51 and SEQ ID NO:52;

nucleotide sequences respectively coding a heavy chain and a light chain of a monoclonal antibody M1, as set forth in SEQ ID NO:53 and SEQ ID NO:54;

nucleotide sequences respectively coding a heavy chain and a light chain of the monoclonal antibody M2, as set forth in SEQ ID NO:55 and SEQ ID NO:56;

nucleotide sequences respectively coding a heavy chain and a light chain of a monoclonal antibody M3, as set forth in SEQ ID NO:57 and SEQ ID NO:58; or

nucleotide sequences respectively coding a heavy chain and a light chain of a monoclonal antibody C1, as set forth in SEQ ID NO:59 and SEQ ID NO:60.

In a third aspect, the invention provides a use of the abovementioned monoclonal antibody in (1) or (2):

(1) detecting PVY;

(2) preparing products for detecting PVY.

Preferably, the products for detecting PVY comprises, but is not limited to, an ELISA detection kit, a Western Blot detection kit, a colloid gold detection kit, and a CLIA kit.

In a fourth aspect, the invention provides an ELISA kit for detecting PVY, wherein the ELISA kit comprises the monoclonal antibody mentioned above.

The invention has the following beneficial effects:

The monoclonal antibodies N1, M1, M2, M3 and C1 and a combination thereof can recognize all or most PVY isolates which have been reported, thus reducing the possibility of detection omissions; and the five monoclonal antibodies do not react with other congeneric viruses and non-congeneric viruses, thus reducing the possibility of detection errors. Therefore, the monoclonal antibodies and the combination thereof can realize accurate PVY detection.

### BRIEF DESCRIPTION OF THE SEVERAL VIEWS OF THE DRAWINGS

FIG. 1: screening, by dot-ELISA, monoclonal antibodies (A) capable of recognizing different regions of CP and identifying antigenic determinants of the monoclonal antibodies.

FIG. 2: analyzing, by dot-ELISA, the recognition of monoclonal antibodies to different PVY isolates.

FIG. 3: comparing antigenic determinant sequences of monoclonal antibodies with corresponding sequences in different PVY CP amino acids.

FIG. 4: analyzing the specificity of monoclonal antibodies to different viruses.

FIG. 5: comparing antigenic determinant sequences of monoclonal antibodies with corresponding sequences in CP amino acids of different viruses of potyvirus.

FIG. 6: analyzing, by dot-ELISA, the antigen sensitivity of monoclonal antibodies.

### DETAILED DESCRIPTION OF THE INVENTION

It should be pointed out that all the following details are merely illustrative and aim to provide a further description of the invention. Unless otherwise stated, all technical and scientific terms used in this specification have the same meaning as commonly understood by those ordinarily skilled in the art.

As mentioned above, detection omissions or errors of PVY may lead to incorrect detection results, are disadvantageous to effective control of PVY, and severely impair safety production of crops. As for PVY detection, PVY polyclonal antibodies can detect other viruses of potyvirus, so detection errors will be caused when polyclonal antibodies are used for PVY detection; commercial PVY monoclonal antibodies Mab1128, Mab1129 and Mab1130 can recognize antigenic determinants ²⁵NLNKEK³⁰, ¹⁶RPEQGSIQSNP²⁶ and ^{5I}DAGGS¹⁰ of coat protein (CP), but many PVY isolates do not include these three antigenic determinants, so detection omissions will be caused when these three monoclonal antibodies are used for PVY detection.

In view of this, in order to prevent detection omissions or errors, it is necessary to screen out PVY monoclonal antibodies capable of recognizing new antigenic determinants, specify the conservativeness and specificity of antigenic determinants, and combine the PVY monoclonal antibodies to realize accurate virus detection.

The invention provides five monoclonal antibodies N1, M1, M2, M3 and C1, antigenic determinants of which are ⁴TIDAGGSTK¹², ³⁷GTSGTHTVP⁴⁵, ⁸⁹QFDTWYE⁹⁵, ¹⁵⁴PTLRQIM¹⁶⁰ and ²⁶¹LLGVKN²⁶⁶ respectively. Up to Apr. 12, 2022, complete CP amino acid sequences of 1,885 PVY isolates are recorded in NCBI totally. An analysis of the antigenic determinants indicates that N1 can recognize at least 694 isolates, M1 can recognize at least 1,853 isolates, M2 can recognize at least 1,843 isolates, M3 can recognize at least 1,848 isolates, and C1 can recognize at least 1,851 isolates; a combination of M1 and M2 can recognize all the 1,885 isolates, a combination of M1 and C1 can recognize 1,883 isolates, and a combination of M2 and C1 can recognize 1,876 isolates. The five monoclonal antibodies will not react with other congeneric viruses and non-congeneric viruses. Therefore, the monoclonal antibodies and the combination thereof provided by the invention can realize accurate detection of PVY.

To allow those skilled in the art to gain a better understanding of the technical solution of the application, the technical solution of the invention will be described in detail below in conjunction with specific embodiments.

All test materials used in the embodiments of the invention are conventional test materials in the art, and are commercially available. The PVY isolate used in the invention has an accession number of X977895 in NCBI. PVY monoclonal antibodies Mab1128, Mab1129 and Mab1130 are recorded in Literature Analysis of Potato virus Y Coat Protein Epitopes Recognized by Three Commercial Monoclonal Antibodies (PLOS ONE, 2014,9(12):e115766).

### Embodiment 1: Preparation of PVY Monoclonal Antibodies Capable of Recognizing Different Epitopes

### 1. Extraction of PVY Particles

200 g of potato leaves infected with PVY and 400 mL of 0.1M phosphate buffer (containing 0.15% β-mercaptoethanol and 0.01M EDTA, pH=7.2) were added into a tissue crusher and stirred for 10 min. Homogenate was filtered with a gauze to remove insoluble plant tissue. An obtained solution was centrifuged at 4° C. and 8000 rpm for 30 min, and supernate was added into 1% Triton-100 and stirred at 4° C. for 3 hrs; and 6% PEG6000 and 1% NaCl were added, and stirred at 4° C. for 3 hrs. The solution was centrifuged at 4° C. and 8000 rpm for 30 min, supernate was removed, precipitate was dissolved in 40 mL of 0.1M phosphate buffer (containing 1% Triton-100, pH=7.2) and stirred at 4° C. overnight. Then, the solution was centrifuged at 4° C. and 8000 rpm for 10 min, precipitate was removed, and supernate was added into a centrifuge tube containing a 30% sucrose cushion, and was ultra-centrifuged at 4° C. and 100,000 g for 2 hrs. Obtained precipitate was dissolved in 2 mL of 0.1M phosphate buffer, and then PVT particles were extracted.

### 2. Mouse Immunization

Purified PVY particles were injected into six-weeks-old female BALB/c mice every three week for three times of immunization in total, and 100 μL (25 μg) of PVY particles were injected into the mice every time. An immunogen for the first time of immunization was emulsified with the same volume of Freund's complete adjuvant, an immunogen for the second time of immunization was emulsified with the same volume of Freud's incomplete adjuvant, and an immunogen for the third time of immunization was mixed with the same volume of normal saline.

### 3. Preparation of Hybridoma Cell Strains

Spleen cells of the BALB/c mice and myeloma cells were mixed according to a ratio of 10:1. 50% PEG 4000 was added to the cells and blended with the cells for 3 min. Then, the cells were washed with a culture solution, then added with HAT cell culture fluid, and placed in a 96-hole cell culture plate. 7 days later, positive cells were screened out through an indirect method ELISA. The positive cells were cultured in a 24-hole plate. Monoclonal cell strains were obtained through a limiting dilution method.

### 4. Mass Preparation of Monoclonal Antibodies

0.5 mL of liquid paraffin was injected into the abdomen of six-weeks-old female BALB/c mice, and one week later, 10⁶ hybridoma cells were injected into the abdomen of the mice. One week later, ascites was collected in time according to abdomen expansion of the mice. Monoclonal antibodies were purified with a Protein G affinity column.

### 5. Screening of Monoclonal Antibodies Capable of Recognizing Different Antigenic Determinants

To obtain PVY monoclonal antibodies capable of recognizing different antigenic determinants, a prokaryotic expression vector pEHISTEV-PVY CP of CP was constructed first (an expression product was PVY CP). Then, deletion mutation was carried out on pEHISTEV-PVY CP, and codon sequences of amino acids 2-15, 2-30, 16-45, 31-60, 46-75, 61-90, 61-255, 226-255, 241-267 and 256-267 were deleted to obtain plasmids of expressible deletion mutants CPΔ2-15, CPΔ2-30, CPΔ16-45, CPΔ31-60, CPΔ46-75, CPΔ61-90, CPΔ61-255, CPΔ226-255, CPΔ241-267 and CPΔ256-267. pEHISTEV-GFP is negative control, and can be expressed as GFP.

dot-ELISA was carried out on the purified monoclonal antibodies, PVY CP and mutants of the PVY CP as follows:

Potato samples infected with PVY were diluted with a PBS buffer according to a weight ratio of 1:40, 2 μL of diluted sample protein was dropwise added on a nitrocellulose membrane (NC membrane), and then the NC membrane was aired at room temperature. The NC membrane was placed in 10 mL of blocking buffer, and was blocked in a table concentrator at room temperature for 2 h or was blocked at 4° C. overnight. The blocking buffer on the NC membrane is washed away, then the NC membrane was placed in skim milk powder containing the purified monoclonal antibodies (primary antibodies) and prepared from TBST, and incubated in the table concentrator at room temperature for 1 h or incubated in a refrigerator at 4° C. overnight, and then the NC membrane was washed with 10 mL of TBST for three times, 10 min each time. The NC membrane was placed in a buffer containing IgG (Sigma-Aldrich) second antibodies labelled by horseradish peroxidase, and incubated in the table concentrator at room temperature for 1 h; the NC membrane was washed with 10 mL of TBST twice, each 10 min, and then washed with TBS for 10 min. A developing agent was added to the NC membrane, and then the MC membrane was placed on a chemiluminescence instrument to be developed and photographed.

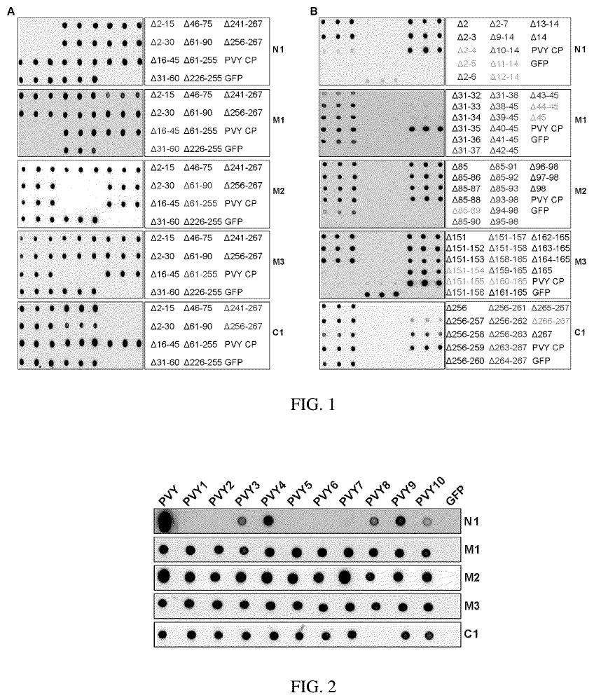

Five monoclonal antibodies N1, M1, M2, M3 and C1 capable of recognizing different areas were screened out according to mutants recognized by the monoclonal antibodies. Wherein, N1 did not react with CPΔ2-15 and CPΔ2-30, M1 did not react with CPΔ16-45 and CPΔ31-60, M2 did not react with CPΔ61-90 and CPΔ61-255, M3 did not react with CPΔ61-255, and C1 did not react with CPΔ241-267 and CPΔ256-267 (FIG. 1A).

### 6. Identification of Antigenic Determinants of the Five PVY Monoclonal Antibodies

Key amino acids were identified according to reaction results to figure out the minimum determinant capable of being recognized by the antibodies. dot-ELISA results indicate that N1 can recognize amino acids 2-15, M1 can recognize amino acids 31-45, M2 can recognize amino acids 61-90, M3 can recognize amino acids 90-226, and C1 can recognize amino acids 256-267. To narrow the range of the regions of antigenic determinants of M2 and M3, a further deletion mutation analysis was carried out to conclude that the antigenic determinant of M2 corresponds to amino acids 85-98, and the antigenic determinant of M3 corresponds to amino acids 151-165.

To identify specific amino acid sites of a PVY CP antigenic determinant recognized by the monoclonal antibody N1, deletion mutation was carried out on amino acids 2-14 of pEHISTEV-PVY CP to obtain plasmids of expressible deletion mutants CPΔ2, CPΔ2-3, CPΔ2-4, CPΔ2-6, CPΔ2-7, CPΔ9-14, CPΔ10-14, CPΔ11-14, CPΔ12-14, CPΔ13-14 and CPΔ14.

dot-ELISA was carried out on the mutants by N1. Results show that N1 reacts with CPΔ2-3 and CPΔ13-14, slightly reacts with CPΔ2-4, CPΔ2-5, CPΔ11-14 and CPΔ12-14, and does not react with CPΔ2-6, CPΔ2-7, CPΔ9-14 and CPΔ10-14 (FIG. 1B), indicating that the antigenic determinant recognized by NI corresponds to amino acids 4-12 TIDAGGSTK of the PVY CP.

According to the range of amino acids recognized by the other four antibodies, deletion mutation was carried out in the similar way to obtain corresponding plasmids. dot-ELISA results show that the monoclonal antibody M1 react with CPΔ31-36, slightly reacts with CPΔ44-45 and CPΔ45, and does not react with CPΔ31-37, CPΔ31-38, CPΔ38-45, CPΔ39-45, CPΔ40-45, CPΔ41-45, CPΔ42-45 and CPΔ43-45 (FIG. 1B), indicating that the antigenic determinant recognized by M1 corresponds to amino acids 37-45 GTSGTHTVP of the PVY CP.

The monoclonal antibody M2 reacts with CPΔ85-88 and CPΔ96-98, slightly reacts with CP485-89, and does not react with CPΔ85-90, CPΔ85-91, CPΔ85-92, CPΔ85-93, CPΔ93-98, CPΔ94-98 and CPΔ95-98 (FIG. 1B), indicating that the antigenic determinant recognized by MI corresponds to amino acids 89-95 QFDTWYE of the PVY CP.

The monoclonal antibody M3 reacts with CPΔ151-153 and CPΔ161-165, slightly reacts with CPΔ151-154, CPΔ151-155 and CPΔ160-165, and does not react with CPΔ151-156, CPΔ151-157, CPΔ151-158, CPΔ158-165 and CPΔ159-165 (FIG. 1B), indicating that the antigenic determinant recognized by N1 corresponds to amino acids 154-160 PTLRQIM of the PVY CP.

The monoclonal antibody C1 reacts with CPΔ256-260 and CPΔ267, slightly reacts with CPΔ266-267, and does not react with CPΔ256-261, CPΔ256-262, CPΔ256-263, CPΔ263-267, CPΔ264-267 and CPΔ265-267 (FIG. 1B), indicating that the antigenic determinant recognized by C1 corresponds to amino acids 261-266 LLGVKN of the PVY CP.

### 7. Identification of the Subtype and Valence of the Five PVY Monoclonal Antibodies

The subtype and subclass of the five monoclonal antibodies MAb N1, M1, M2, M3 and C1 are identified as IgG1, IgG1, IgG1, IgG1 and IgG2b, with a light chain kappa (Table 1). By indirect ELISA detection, the valence of the five monoclonal antibodies can reach 1:243000 (Table 1).

TABLE 1
Subtype and valence of monoclonal antibodies

| MAbs | Isotype | Titer |
|---|---|---|
| N1 | IgG1, κ chain | 1:243000 |
| M1 | IgG1, κ chain | 1:243000 |
| M2 | IgG1, κ chain | 1:243000 |
| M3 | IgG1, κ chain | 1:243000 |
| C1 | IgG2b, κ chain | 1:243000 |

### Embodiment 2: Detection of PVY Isolate CP that Cannot be Recognized by MAb1128, MAb1129 and MAb1130 with MAbs N1, M1, M2, M3 and C1

10 PVY isolate CP sequences not including antigenic determinants that can be recognized by MAb1128, MAb1129 and MAb1130 were synthesized. The serial numbers of 10 PVY isolates in NCBI were CAA66472, CAA48304, ABY78978, CAA48306, CAA94183, AAL35616, ABQ53158, CAE51190, CAA32356, CAA48302 and CAA94184 respectively.

dot-ELISA results indicate that N1 cannot recognize isolates PVY1, PVY2, PVY5 and PVY6, M1,M2 and M3can recognize all the 10 isolates, and C1 cannot recognize the isolate PVY8 (FIG. 2).

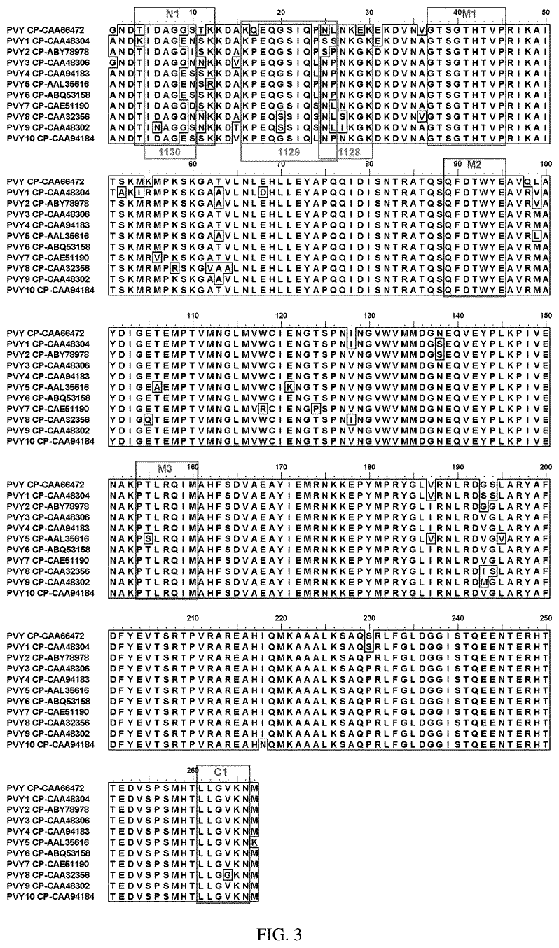

It is found by a further sequence analysis that the 10 PVY isolate CP sequences do not include the antigenic determinant recognized by N1 and include antigenic determinants recognized by M1 and M2, the other 9 isolate CP sequences except PVY5 include the antigenic determinant recognized by M3, and the other 9 isolate CP sequences except PVY8 include the antigenic determinant recognized by C1 (FIG. 3), indicating that M1, M2, M3 and C1 can recognize all or most isolates that cannot be recognized by MAb1128, MAb1129 and MAb1130.

### Embodiment 3: Specificity Analysis of the PVY Monoclonal Antibodies

Specificity analysis was carried out on the five monoclonal antibodies N1, M1, M2, M3 and C1 screened out in Embodiment 1 specifically as follows:

The five monoclonal antibodies were used for detecting, by means of dot blot, prokaryotic expression CPs of turnip mosaic virus (TuMV) (genus Potyvirus), tobacco vein banding mosaic virus (TVBMV) (genus Potyvirus), potato virus X (PVX) (genus Potexvirus), tobacco mosaic virus (TMV) (genus Tobamovirus), tomato mosaic virus (ToMV) (genus Tobamovirus), tomato mottle mosaic virus (ToMMV) (genus Tobamovirus), tomato brown rugose fruit virus (ToMMV) (genus Tobamovirus), chilli ringspot virus (ChiRSV) (genus Potyvirus), chilli mottle virus (PepMoV) (genus Potyvirus), potato virus A (PVA) (genus Potyvirus), chilliveinal mottle virus (PVMV) (genus Potyvirus), tobacco vein mottling virus (TVMV) (genus Potyvirus), potato virus M (PVM) (genus Carlavirus), potato virus M (PVM) (genus Carlavirus), and potato virus S (PVS) (genus Carlavirus).

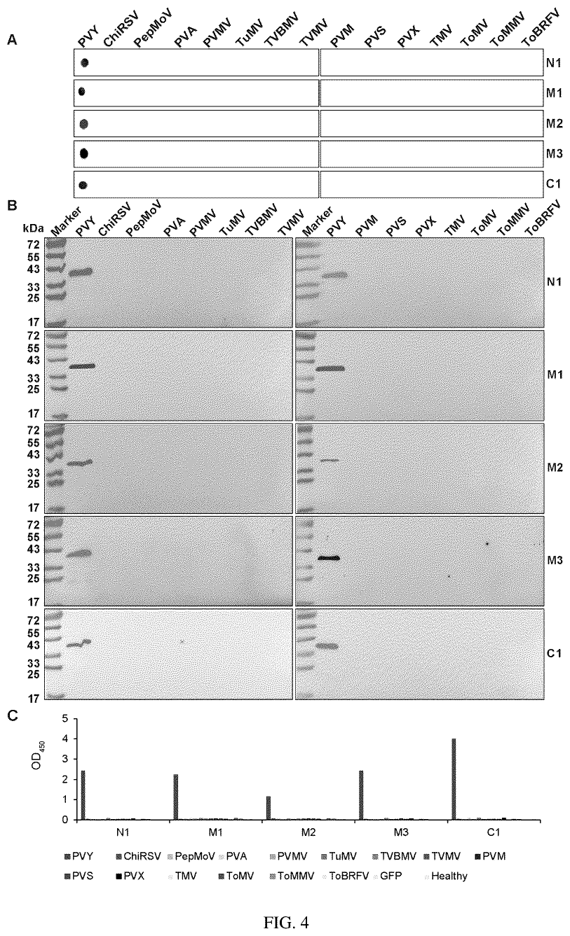

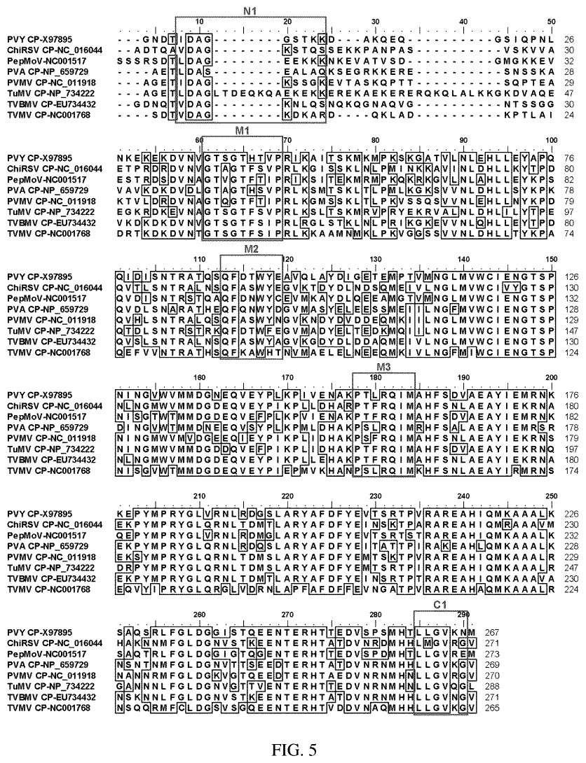

Dot blot results show that the five monoclonal antibodies can recognize PVY and will not undergo a cross reaction with the other 14 viruses or CPs, thus having high specificity (FIG. 4A). Western blot and ELISA also further prove that the specificity of the five monoclonal antibodies (FIG. 4B and FIG. 4C). It is found, by analyzing the CP sequences of the eight viruses of Potyvirus, that the antigenic determinant sequences of the five monoclonal antibodies are not 100% consistent with the sequences of the other seven viruses (FIG. 5), indicating that the five monoclonal antibodies can specially recognize antigenic determinant sequences on the PVY CP.

### Embodiment 4: Sensitivity Analysis of the PVY Monoclonal Antibodies

Sensitivity analysis was carried out on the five monoclonal antibodies N1, M1, M2, M3 and C1 screened out in Embodiment 1 respectively through dot-ELISA.

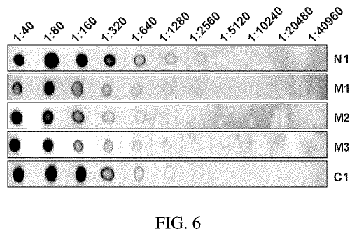

First, juice infected with PVY was diluted. Then, dot-ELISA was carried out in the same way as Embodiment 1. Results indicate that N1 can detect the juice diluted by 10,240 times, M1 can detect the juice diluted by 5,120 times, M2 can detect the juice diluted by 640 times, M3 can detect the juice diluted by 5,120 times, and C1 can detect the juice diluted by 5,120 times (FIG. 6).

### Embodiment 5: Analysis of PVY Isolate Coverage of the PVY Monoclonal Antibodies

Up to Apr. 12, 2022, the complete CP amino acid sequences of 1,885 PVY isolates are recorded in NCBI totally. It was found, by sequence analysis, that 694 isolates include the antigenic determinant sequence TIDAGGSTK recognized by N1, 1,853 isolates include the antigenic determinant sequence GTSGTHTVP recognized by M1, 1,843 isolates include the antigenic determinant sequence QFDTWYE recognized by M2, 1,848 isolates include the antigenic determinant sequence PTLRQIM recognized by M3, and 1,851 isolates include the antigenic determinant sequence LLGVKN recognized by C1. The monoclonal antibodies M1, M2, M3 and C1 can detect 11.35%, 10.82%, 11.09% and 11.25% more isolates than MAb1130 (Table 2).

TABLE 2
PVY isolate coverage of monoclonal antibodies

| | The number (percentage) | | The number (percentage) | |
|---|---|---|---|---|
| | of isolates without the | | of isolates with the | |
| | epitope among 1885 | | epitope among 1885 | |
| MAbs | isolates | | isolates | |
| 1128 | 1146 | (60.80%) | 739 | (39.20%) |
| 1129 | 1238 | (65.68%) | 647 | (34.32%) |
| 1130 | 246 | (13.05%) | 1639 | (86.95%) |
| N1 | 1191 | (63.18%) | 694 | (36.82%) |
| M1 | 32 | (1.70%) | 1853 | (98.3%) |
| M2 | 42 | (2.23%) | 1843 | (97.77%) |
| M3 | 37 | (1.96%) | 1848 | (98.04%) |
| C1 | 34 | (1.80%) | 1851 | (98.20%) |

The antigenic determinant (PTLRQIM) recognized by the monoclonal antibody M3 corresponds to the sequence PSLRQIM in the PVY5 isolate CP, and also corresponds to the sequence PSLRQIM in TVMV and PVA CP. As analyzed in Embodiment 2 and Embodiment 3, although M3 can recognize PVY5, it cannot recognize TVMV and PVA, so M3 is not the optimal antibody, and will not be taken into account by preferred PVY monoclonal antibody combinations. In addition, the monoclonal antibody N1 can only recognize a few isolates, and thus will not be taken into account either. The 1,885 PVY isolates are numbered from 1-1885, and it can be found, by analyzing the antigenic determinants of the PVY isolates, that themonoclonal antibody M1 cannot recognize 32 PVY isolates, the monoclonal antibody M2 cannot recognize 42 PVY isolates, and the monoclonal antibody C1 cannot recognize 34 PVY isolates. The serial numbers of the isolates are specially shown in Table 3. It is found, by analyzing the serial numbers of the PVY isolates not including the antigenic determinants recognized by MAb M1, M2or C1, that a combination of the monoclonal antibodies M1 and M2 can recognize all the 1,885 PVY isolates, a combination of the monoclonal antibodies M1 and C1 can recognize the other 1,883 PVY isolates except 1107 (NCBIaccession number in NCBI: AMW64585.1) and 1344(AYA57928.1), a combination of the monoclonal antibodies M2 and C1 can recognize the other 1,876 PVY isolates except 380(AEF59038.1), 381 (AEF59039.1), 788(AGX27987.1), 789(AGX27988.1), 790(AGX27989.1), 991(AIL50122.1), 992(AIL50123.1), 993(AIL50124.1) and 994(AIL50125.1).

### Embodiment 6: Sequence and Structure Analysis of the PVY Monoclonal Antibodies

Sequence and structure analysis was further carried out on the five monoclonal antibodies N1, M1, M2, M3 and C1 screened out in Embodiment 1, wherein:

The monoclonal antibodies comprise: a heavy chain and a light chain. Specifically:

An amino acid sequence of the heavy chain of the monoclonal antibody N1 is:

MNLGLSFIFLALILKGVQCEVQLVESGGGLVQPGGSLKLSCAASGFTFS [symbol]
WVRQSPDKRLEWVA [symbol] [symbol] RFTISRDNAKN
TLYLQMSSLKSEDTAVYYCAR [symbol] WGQGTTLTVSS

Note: the shadow area is a heavy-chain variable region; the italic, bold and underlined areas are CDRs, which are VHCDR1, VHCDR2 and VHCDR3 in sequence.

An amino acid sequence of the light chain of the monoclonal antibody N1 is:

MDSQAQVLILLLLWVSGTCGDIVMSQSPSSLAVSAGEKVTMSC [symbol] [symbol] [symbol]
WYQQKPGQSPKLLIY [symbol] GVPDRFTGSGSGTD
FTLTISSVQAEDLAVYYC [symbol] FGSGTKLEIK

Note: the shadow area is a light-chain variable region; the italic, bold and underlined areas are CDRs, which are VLCDR1, VLCDR2 and VLCDR3 in sequence.

A nucleotide sequence for coding the heavy chain of the monoclonal antibody N1 is:

ATGAACTTAGGGCTCAGCTTCATTTTCCTTGCCCTTATTTTAAAAGGTG
TCCAGTGTGAGGTGCAACTGGTGGAGTCTGGGGGAGGCTTAGTGCAGCCT
GGAGGGTCCCTGAAACTCTCCTGTGCAGCCTCTGGATTCACTTTCAGTAG
CTATGGCATGTCTTGGGTTCGCCAGAGTCCAGACAAGAGGCTGGAATGGG
TCGCAACCATTAATAATAATGGTGGTAGCACCTATTATCCAGACAGTGTG
AAGGGCCGATTCACCATCTCCAGAGACAATGCCAAGAACACCCTGTACCT
GCAAATGAGCAGTCTGAAGTCTGAGGACACAGCCGTGTATTACTGTGCAA
GAGATGCGGCCTTTGACTGCTGGGGCCAAGGCACCACTCTCACAGTCTCC
TCA

Note: the shadow area is a coding sequence of the heavy-chain variable region.

A nucleotide sequence for coding the light chain of the monoclonal antibody N1 is:

ATGGATTCACAGGCCCAGGTTCTTATATTGCTGCTGCTATGGGTATCTG
GTACCTGTGGGGACATTGTGATGTCACAGTCTCCATCCTCCCTGGCTGTG
TCAGCAGGAGAGAAGGTCACTATGAGCTGCAAATCCAGTCAGAGTCTGCT
CAACAGTAGAACCCGAAAGAACTACTTGGCTTGGTACCAGCAGAAACCAG
GGCAGTCTCCTAAACTGCTGATCTACTGGGCATCCACTAGGGAATCTGGG
GTCCCTGATCGCTTCACAGGCAGTGGATCTGGGACAGATTTCACTCTCAC
CATCAGCAGTGTGCAGGCTGAGGACCTGGCAGTTTATTACTGCAAGCAAT
CTTATAATCTATTCACGTTCGGCTCGGGGACAAAGTTGGAAATAAAA

Note: the shadow area is a coding sequence of the light-chain variable region.

An amino acid sequence of the heavy chain of the monoclonal antibody M1 is:

MGWSCIILFLAATATGVHSQVQLQQSGPELVRPGASVKMSCKTSGYPFS [symbol] [symbol]
WVKQRPGQGLEWIG [symbol] [symbol] KATLNVDKS
SNTAYMQLNSLTSEDSGVYYCAR [symbol] WGQGTTLTVSS

Note: the shadow area is a heavy-chain variable region; the italic, bold and underlined areas are CDRs, which are VHCDR1, VHCDR2 and VHCDR3 in sequence.

An amino acid sequence of the light chain of the monoclonal antibody M1 is:

MSPAQFLILLVLWIRETNGDVVLTQTPLTLSVTIGQPASISC [symbol] [symbol]
WLLQRPGQSPKRLIY [symbol]
GVPDRFTGSGSGTDFTLKISRVEADDLGIYYC [symbol]
FGAGTKLELK

Note: the shadow area is a light-chain variable region; the italic, bold and underlined areas are CDRs, which are VLCDR1, VLCDR2 and VLCDR3 in sequence.

A nucleotide sequence for coding the heavy chain of the monoclonal antibody M1 is:

ATGGGATGGAGCTGTATCATCCTCTTCTTGGCAGCAACAGCTACAGGT
GTCCACTCCCAGGTCCAACTGCAGCAGTCTGGGCCTGAGCTGGTGAGGCC
TGGGGCTTCAGTGAAGATGTCCTGTAAGACTTCAGGCTATCCCTTCAGCA
CCTACTGGATGCACTGGGTGAAACAGAGGCCTGGACAAGGCCTTGAATGG
ATTGGCATGATTAAACCTTCCAATAGTGAAACTAGTTTAAATCAGAAATT
CAAGGACAAGGCCACATTGAATGTAGACAAATCCTCCAATACAGCCTACA
TGCAGCTCAACAGCCTGACATCTGAGGACTCTGGAGTCTATTACTGTGCA
AGGGCAGGGGACTCCTGGGGCCAGGGCACCACTCTCACAGTCTCCTCA

Note: the shadow area is a coding sequence of the heavy-chain variable region.

A nucleotide sequence for coding the light chain of the monoclonal antibody M1 is:

ATGAGTCCTGCCCAGTTCCTGATTCTGTTAGTGCTCTGGATTCGGGAA
ACCAACGGTGATGTTGTGCTGACCCAGACTCCACTCACTTTGTCGGTTAC
CATTGGACAACCAGCCTCCATCTCTTGCAAGTCAAGTCAGAGCCTCTTAG
AAACTGATGGACAGACATTTTTGAATTGGTTGTTACAGCGGCCAGGCCAG
TCTCCAAAGCGCCTAATCTATCTGGTGTCTAAACTGGACTCTGGAGTCCC
TGACAGGTTCACTGGCAGTGGATCAGGGACAGATTTCACACTGAAAATCA
GCAGAGTGGAGGCTGACGATTTGGGAATTTATTATTGCTGGCAAGGTACA
CATTTTCCGCTCACGTTCGGTGCTGGGACCAAGCTGGAGCTGAAA

Note: the shadow area is a coding sequence of the light-chain variable region.

An amino acid sequence of the heavy chain of the monoclonal antibody M2 is:

MLLGLKWVFFVVFYQGVHCEVOLVESGGGLVQPKGSLKLSCAASDFTF
N [symbol] WVRQAPGKVLEWIA [symbol] [symbol] RFTISRDD
SQNMFYLQMNNLKTEDTAMYYCAG [symbol] WGQGTLVAVSA

Note: the shadow area is a heavy-chain variable region; the italic, bold and underlined areas are CDRs, which are VHCDR1, VHCDR2 and VHCDR3 in sequence.

An amino acid sequence of the light chain of the monoclonal antibody M2 is:

MKLPVRLLVLMFWIPASSSDVLMTQTPLSLPVSLGDQASISC [symbol] [symbol]
WYLQKPGQSPKLLIY [symbol] GVPDRFSGS
GSGTDFTLKISRVEAEDLGVYFC [symbol] FGGGTKLEIK

Note: the shadow area is a light-chain variable region; the italic, bold and underlined areas are CDRs, which are VLCDR1, VLCDR2 and VLCDR3 in sequence.

A nucleotide sequence for coding the heavy chain of the monoclonal antibody M2 is:

ATGCTGTTGGGGCTGAAGTGGGTTTTCTTTGTTGTTTTTTATCAAGGTG
TGCATTGTGAGGTGCAGCTTGTTGAGTCTGGTGGAGGATTGGTGCAGCC
TAAAGGGTCATTGAAACTCTCATGTGCAGCCTCTGATTTCACCTTCAAT
ACCCACTCCATGAACTGGGTCCGCCAGGCTCCAGGAAAGGTTTTGGAAT
GGATTGCTCGCATAAGAAGTCAAAGTAATAATTATGCAACATATTATGC
CGATTCAGTGAAAGACAGGTTCACCATCTCCAGAGATGATTCACAAAAC
ATGTTCTATCTGCAAATGAACAACTTGAAAACTGAGGACACAGCCATGT
ATTACTGTGCTGGGGATTACTACGGCCCCTTTGCTTACTGGGGCCAAGG
GACTCTGGTCGCTGTCTCTGCA

Note: the shadow area is a coding sequence of the heavy-chain variable region.

A nucleotide sequence for coding the light chain of the monoclonal antibody M2 is:

ATGAAGTTGCCTGTTAGGCTGTTGGTGCTGATGTTCTGGATTCCTGCTT
CCAGCAGTGATGTTCTGATGACCCAAACTCCACTCTCCCTGCCTGTCAG
TCTTGGAGATCAAGCCTCCATCTCTTGCAGATCTGGTCAGAGCCTTGTA
CACAGTAATGGAAACACCTATTTACATTGGTACCTGCAGAAGCCAGGCC
AGTCTCCAAAGCTCCTGATCTACAAAGTTTCCAACCGATTTTCTGGGGT
CCCAGACAGGTTCAGTGGCAGTGGATCAGGGACAGATTTCACACTCAAG
ATCAGCAGAGTGGAGGCTGAGGATCTGGGAGTTTATTTCTGCTCTCAAA
GTACACATGTTCCGTGGACGTTCGGTGGAGGCACCAAGCTGGAAATCAA
A

Note: the shadow area is a coding sequence of the light-chain variable region.

An amino acid sequence of the heavy chain of the monoclonal antibody M3 is:

MNFGLSLIFLVLVLKGVQCEVILVESGGGLVKPGGSLRLSCAASGFTFS [symbol] [symbol]
WVRQSPDKRLEWV [symbol] [symbol] RLTISRDNAKNIL
YLHMNSLRSEDTAMYYCAR [symbol] WGQGTLVTVSA

Note: the shadow area is a heavy-chain variable region; the italic, bold and underlined areas are CDRs, which are VHCDR1, VHCDR2 and VHCDR3 in sequence.

An amino acid sequence of the light chain of the monoclonal antibody M3 is:

METDTILLWVLLLWVPGSTGDIALTQSPASLAVSLGQRATISC [symbol] [symbol]
WYQQKPGQPPKLLIY [symbol] GIPARFSGSGS
GTDFTLNIHPVEEEDAATYYC [symbol] FGGGTKLEIK

Note: the shadow area is a light-chain variable region; the italic, bold and underlined areas are CDRs, which are VLCDR1, VLCDR2 and VLCDR3 in sequence.

A nucleotide sequence for coding the heavy chain of the monoclonal antibody M3 is:

ATGAACTTCGGGCTCAGCTTGATTTTCCTTGTCCTTGTTTTAAAAGGTG
TCCAGTGTGAGGTGATACTGGTGGAGTCTGGGGGGGGCTTAGTGAAGCC
TGGAGGGTCCCTGAGACTCTCCTGTGCAGCCTCTGGATTCACTTTCCAG
TAGCATCCCTTGTCTTGGGTTCGCCAGAGTCCGGACAAGAGGCTGGAAT
GGGTCGCAAGCATTAGTAGTGGTGGTAGGTACACCTACTATCCAGACAG
TGTGAAGGGGCGACTCACCATCTCCAGAGACAATGCCAAGAACATCCTG
TATTTACACATGAACAGTCTGAGGTCTGAGGACACGGCCATGTATTACT
GTGCAAGACGGGGCTCTACGGCTACGGCCTGGTTTGCTTACTGGGGCCA
AGGGACTCTGGTCACTGTCTCTGCA

Note: the shadow area is a coding sequence of the heavy-chain variable region.

A nucleotide sequence for coding the light chain of the monoclonal antibody M3 is:

ATGGAGACAGACACAATCCTGCTATGGGTGCTGCTGCTCTGGGTTCCA
GGCTCCACTGGTGACATTGCGCTGACCCAATCTCCAGCTTCTTTGGCTG
TGTCTCTAGGGCAGAGGGCCACCATCTCCTGCAAGGCCAGCCAAAGTGT
TGATTATGATGGTGATAGTTATATGAGCTGGTACCAACAGAAACCAGGA
CAGCCACCCAAACTCCTCATCTATGGTGCATCCAATCTAGAATCTGGGA
TCCCAGCCAGGTTTAGTGGCAGTGGGTCTGGGACAGACTTCACCCTCAA
CATCCATCCTGTGGAGGAGGAGGATGCTGCAACCTATTACTGTCAGCAA
AGTAATGAGGATCTCCCGACGTTCGGTGGAGGCACCAAGCTGGAAATCA
AA

Note: the shadow area is a coding sequence of the light-chain variable region.

An amino acid sequence of the heavy chain of the monoclonal antibody C1 is:

MKCSWVIFFLMAVVTGVNSEVQLQQSGAELVRPGALVKLSCKASGFNIK [symbol]
WVKQRPEEGLEWIG [symbol] [symbol] KASITADTSSNTV
RLQLSSLTSEDTAVYYCAR [symbol] WGQGTLVTVSA

Note: the shadow area is a heavy-chain variable region; the italic, bold and underlined areas are CDRs, which are VHCDR1, VHCDR2 and VHCDR3 in sequence.

An amino acid sequence of the light chain of the monoclonal antibody C1 is:

MDFQVQIFSFLLISASVILSRGQIVLTQSPAIMSASPGEKVTMTC [symbol] [symbol]
WFQQKPGSSPRLLIYGVPVRFSGSGSGTSY
SLTISRMEAEDAATFYC [symbol] FGAGTKLELK

Note: the shadow area is a light-chain variable region; the italic, bold and underlined areas are CDRs, which are VLCDR1, VLCDR2 and VLCDR3 in sequence.

A nucleotide sequence for coding the heavy chain of the monoclonal antibody C1 is:

ATGAAATGCAGCTGGGTCATCTTCTTCCTGATGGCAGTGGTTACAGGG
GTCAATTCAGAGGTTCAGCTGCAGCAGTCTGGGGCTGAGCTTGTGAGGC
CAGGGGCCTTAGTCAAGTTGTCCTGCAAAGCATCTGGCTTCAACATTAA
AGACTACTATATGCACTGGGTGAAGCAGAGGCCTGAAGAGGGCCTGGAG
TGGATTGGATGGATTGATCCTGAGAATGGTAATACTATATATGACCCGA
AGTTCCAGGGCAAGGCCAGTATAACAGCAGACACATCCTCCAACACAGT
CAGGCTGCAGCTCAGCAGCCTGACATCTGAGGACACTGCCGTCTATTAC
TGTGCTAGAGAGAGGGGCGCTTACTGGGGCCAAGGGACTCTGGTCACTG
TCTCTGCA

Note: the shadow area is a coding sequence of the heavy-chain variable region.

A nucleotide sequence for coding the light chain of the monoclonal antibody C1 is:

ATGGATTTTCAAGTGCAGATTTTCAGCTTCCTGCTAATCAGTGCCTCAG
TCATACTGTCCAGAGGACAAATTGTTCTCACCCAGTCTCCAGCAATCAT
GTCTGCATCTCCAGGGGAGAAGGTCACCATGACCTGCAGTGCCAGCTCA
AGTGTAAGTTACATGTACTGGTTCCAGCAGAAGCCAGGATCCTCCCCCA
GACTCCTGATTTATGACACATCCAACCTGGCTTCTGGAGTCCCTGTTCG
CTTCAGTGGCAGTGGGTCTGGGACCTCTTACTCTCTCACAATCAGCCGA
ATGGAGGCTGAAGATGCTGCCACTTTTTACTGCCAGCAGTGGAGTAGTT
ACCCGCTCACGTTCGGTGCTGGGACCAAGCTGGAGCTGAAA

Note: the shadow area is a coding sequence of the light-chain variable region.

The monoclonal antibodies N1, M1, M2, M3 and C1 obtained in the invention have specific CDRs, and are significantly different from existing reported monoclonal antibodies. The five monoclonal antibodies and a combination thereof can recognize all or most PVY isolates which have been reported, thus reducing the possibility of detection omissions; and the five monoclonal antibodies do not react with other congeneric viruses and non-congeneric viruses, thus reducing the possibility of detection errors. Therefore, the monoclonal antibodies and the combination thereof can realize accurate PVY detection.

The above embodiments are merely preferred ones of the application, and are not used to limit the invention. For those skilled in the art, various modifications and transformations of the application can be made. Any modifications, equivalent substitutions and improvements made based on the spirit and principle of the application should fall within the protection scope of the application.

1. A monoclonal antibody for detecting PVY, wherein the monoclonal antibody is monoclonal antibodies M1;
the monoclonal antibody M1 has heavy-chain CDRs respectively as set forth in SEQ ID NO:7-9, and light-chain CDRs respectively as set forth in SEQ ID NO: 10-12.

2. The monoclonal antibody according to claim 1, wherein a heavy-chain variable region of the monoclonal antibody M1 has an amino acid sequence as set forth in SEQ ID NO:33, and a light-chain variable region of the monoclonal antibody M1 has an amino acid sequence as set forth in SEQ ID NO:34.

3. The monoclonal antibody according to claim 1, wherein a heavy chain of the monoclonal antibody M1 has an amino acid sequence as set forth in SEQ ID NO:43, and a light chain of the monoclonal antibody M1 has an amino acid sequence as set forth in SEQ ID NO:44.

4. A coding gene of the monoclonal antibody according to claim 3.

5. The coding gene according to claim 4, wherein the coding gene comprises:
nucleotide sequences respectively coding a heavy chain and a light chain of the monoclonal antibody M1, as set forth in SEQ ID NO:53 and SEQ ID NO:54.

6. Combinations of monoclonal antibodies for detecting PVY, wherein the combinations of monoclonal antibodies comprises: monoclonal antibodies M1 according to claim 1, and monoclonal antibodies M2;
the monoclonal antibody M2 has heavy-chain CDRs respectively as set forth in SEQ ID NO:13-15, and light-chain CDRs respectively as set forth in SEQ ID NO:16-18.

7. The combinations of monoclonal antibodies according to claim 6, wherein a heavy-chain variable region of the monoclonal antibody M2 has an amino acid sequence as set forth in SEQ ID NO:35, and a light-chain variable region of the monoclonal antibody M2 has an amino acid sequence as set forth in SEQ ID NO:36.

8. A use of combinations of monoclonal antibodies according to claim 6 in (1) or (2):
(1) detecting PVY;
(2) preparing products for detecting PVY.

9. The use according to claim 8, wherein the products for detecting PVY comprises an ELISA detection kit, a Western Blot detection kit, a colloid gold detection kit, and a CLIA kit.

10. An ELISA kit for detecting PVY, wherein the ELISA kit comprises the combinations of monoclonal antibodies according to claim 6.


SEQUENCE LISTING (60 sequences)

SEQ ID NO: 1 - protein, 5 aa, synthetic construct
SYGMS

SEQ ID NO: 2 - protein, 17 aa, synthetic construct
TINNNGGSTYYPDSVKG

SEQ ID NO: 3 - protein, 6 aa, synthetic construct
DAAFDC

SEQ ID NO: 4 - protein, 17 aa, synthetic construct
KSSQSLLNSRTRKNYLA

SEQ ID NO: 5 - protein, 7 aa, synthetic construct
WASTRES

SEQ ID NO: 6 - protein, 8 aa, synthetic construct
KQSYNLFT

SEQ ID NO: 7 - protein, 5 aa, synthetic construct
TYWMH

SEQ ID NO: 8 - protein, 17 aa, synthetic construct
MIKPSNSETSLNQKFKD

SEQ ID NO: 9 - protein, 4 aa, synthetic construct
AGDS

SEQ ID NO: 10 - protein, 16 aa, synthetic construct
KSSQSLLETDGQTFLN

SEQ ID NO: 11 - protein, 7 aa, synthetic construct
LVSKLDS

SEQ ID NO: 12 - protein, 9 aa, synthetic construct
WQGTHFPLT

SEQ ID NO: 13 - protein, 5 aa, synthetic construct
THSMN

SEQ ID NO: 14 - protein, 19 aa, synthetic construct
RIRSQSNNYATYYADSVKD

SEQ ID NO: 15 - protein, 8 aa, synthetic construct
DYYGPFAY

SEQ ID NO: 16 - protein, 16 aa, synthetic construct
RSGQSLVHSNGNTYLH

SEQ ID NO: 17 - protein, 7 aa, synthetic construct
KVSNRFS

SEQ ID NO: 18 - protein, 9 aa, synthetic construct
SQSTHVPWT

SEQ ID NO: 19 - protein, 5 aa, synthetic construct
SHPLS

SEQ ID NO: 20 - protein, 17 aa, synthetic construct
SISSGGRYTYYPDSVKG

SEQ ID NO: 21 - protein, 11 aa, synthetic construct
RGSTATAWFAY

SEQ ID NO: 22 - protein, 15 aa, synthetic construct
KASQSVDYDGDSYMS

SEQ ID NO: 23 - protein, 7 aa, synthetic construct
GASNLES

SEQ ID NO: 24 - protein, 9 aa, synthetic construct
QQSNEDLPT

SEQ ID NO: 25 - protein, 5 aa, synthetic construct
DYYMH

SEQ ID NO: 26 - protein, 17 aa, synthetic construct
WIDPENGNTIYDPKFQG

SEQ ID NO: 27 - protein, 5 aa, synthetic construct
ERGAY

SEQ ID NO: 28 - protein, 10 aa, synthetic construct
SASSSVSYMY

SEQ ID NO: 29 - protein, 7 aa, synthetic construct
DTSNLAS

SEQ ID NO: 30 - protein, 9 aa, synthetic construct
QQWSSYPLT

SEQ ID NO: 31 - protein, 115 aa, synthetic construct
EVQLVESGGGLVQPGGSLKLSCAASGFTFSSYGMSWVRQSPDKRLEWVATINNNGGSTYYPDSVKGRFTISRDNAKNTLYLQMSSLKSEDTAVYYCARDAAFDCWGQGTTLTVSS

SEQ ID NO: 32 - protein, 112 aa, synthetic construct
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYNLFTFGSGTKLEIK

SEQ ID NO: 33 - protein, 113 aa, synthetic construct
QVQLQQSGPELVRPGASVKMSCKTSGYPFSTYWMHWVKQRPGQGLEWIGMIKPSNSETSLNQKFKDKATLNVDKSSNTAYMQLNSLTSEDSGVYYCARAGDSWGQGTTLTVSS

SEQ ID NO: 34 - protein, 112 aa, synthetic construct
DVVLTQTPLTLSVTIGQPASISCKSSQSLLETDGQTFLNWLLQRPGQSPKRLIYLVSKLDSGVPDRFTGSGSGTDFTLKISRVEADDLGIYYCWQGTHFPLTFGAGTKLELK

SEQ ID NO: 35 - protein, 119 aa, synthetic construct
EVQLVESGGGLVQPKGSLKLSCAASDFTFNTHSMNWVRQAPGKVLEWIARIRSQSNNYATYYADSVKDRFTISRDDSQNMFYLQMNNLKTEDTAMYYCAGDYYGPFAYWGQGTLVAVSA

SEQ ID NO: 36 - protein, 112 aa, synthetic construct
DVLMTQTPLSLPVSLGDQASISCRSGQSLVHSNGNTYLHWYLQKPGQSPKLLIYKVSNRFSGVPDRFSGSGSGTDFTLKISRVEAEDLGVYFCSQSTHVPWTFGGGTKLEIK

SEQ ID NO: 37 - protein, 120 aa, synthetic construct
EVILVESGGGLVKPGGSLRLSCAASGFTFSSHPLSWVRQSPDKRLEWVASISSGGRYTYYPDSVKGRLTISRDNAKNILYLHMNSLRSEDTAMYYCARRGSTATAWFAYWGQGTLVTVSA

SEQ ID NO: 38 - protein, 111 aa, synthetic construct
DIALTQSPASLAVSLGQRATISCKASQSVDYDGDSYMSWYQQKPGQPPKLLIYGASNLESGIPARFSGSGSGTDFTLNIHPVEEEDAATYYCQQSNEDLPTFGGGTKLEIK

SEQ ID NO: 39 - protein, 114 aa, synthetic construct
EVQLQQSGAELVRPGALVKLSCKASGFNIKDYYMHWVKQRPEEGLEWIGWIDPENGNTIYDPKFQGKASITADTSSNTVRLQLSSLTSEDTAVYYCARERGAYWGQGTLVTVSA

SEQ ID NO: 40 - protein, 106 aa, synthetic construct
QIVLTQSPAIMSASPGEKVTMTCSASSSVSYMYWFQQKPGSSPRLLIYDTSNLASGVPVRFSGSGSGTSYSLTISRMEAEDAATFYCQQWSSYPLTFGAGTKLELK

SEQ ID NO: 41 - protein, 134 aa, synthetic construct
MNLGLSFIFLALILKGVQCEVQLVESGGGLVQPGGSLKLSCAASGFTFSSYGMSWVRQSPDKRLEWVATINNNGGSTYYPDSVKGRFTISRDNAKNTLYLQMSSLKSEDTAVYYCARDAAFDCWGQGTTLTVSS

SEQ ID NO: 42 - protein, 132 aa, synthetic construct
MDSQAQVLILLLLWVSGTCGDIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYNLFTFGSGTKLEIK

SEQ ID NO: 43 - protein, 132 aa, synthetic construct
MGWSCIILFLAATATGVHSQVQLQQSGPELVRPGASVKMSCKTSGYPFSTYWMHWVKQRPGQGLEWIGMIKPSNSETSLNQKFKDKATLNVDKSSNTAYMQLNSLTSEDSGVYYCARAGDSWGQGTTLTVSS

SEQ ID NO: 44 - protein, 131 aa, synthetic construct
MSPAQFLILLVLWIRETNGDVVLTQTPLTLSVTIGQPASISCKSSQSLLETDGQTFLNWLLQRPGQSPKRLIYLVSKLDSGVPDRFTGSGSGTDFTLKISRVEADDLGIYYCWQGTHFPLTFGAGTKLELK

SEQ ID NO: 45 - protein, 138 aa, synthetic construct
MLLGLKWVFFVVFYQGVHCEVQLVESGGGLVQPKGSLKLSCAASDFTFNTHSMNWVRQAPGKVLEWIARIRSQSNNYATYYADSVKDRFTISRDDSQNMFYLQMNNLKTEDTAMYYCAGDYYGPFAYWGQGTLVAVSA

SEQ ID NO: 46 - protein, 131 aa, synthetic construct
MKLPVRLLVLMFWIPASSSDVLMTQTPLSLPVSLGDQASISCRSGQSLVHSNGNTYLHWYLQKPGQSPKLLIYKVSNRFSGVPDRFSGSGSGTDFTLKISRVEAEDLGVYFCSQSTHVPWTFGGGTKLEIK

SEQ ID NO: 47 - protein, 139 aa, synthetic construct
MNFGLSLIFLVLVLKGVQCEVILVESGGGLVKPGGSLRLSCAASGFTFSSHPLSWVRQSPDKRLEWVASISSGGRYTYYPDSVKGRLTISRDNAKNILYLHMNSLRSEDTAMYYCARRGSTATAWFAYWGQGTLVTVSA

SEQ ID NO: 48 - protein, 131 aa, synthetic construct
METDTILLWVLLLWVPGSTGDIALTQSPASLAVSLGQRATISCKASQSVDYDGDSYMSWYQQKPGQPPKLLIYGASNLESGIPARFSGSGSGTDFTLNIHPVEEEDAATYYCQQSNEDLPTFGGGTKLEIK

SEQ ID NO: 49 - protein, 133 aa, synthetic construct
MKCSWVIFFLMAVVTGVNSEVQLQQSGAELVRPGALVKLSCKASGFNIKDYYMHWVKQRPEEGLEWIGWIDPENGNTIYDPKFQGKASITADTSSNTVRLQLSSLTSEDTAVYYCARERGAYWGQGTLVTVSA

SEQ ID NO: 50 - protein, 128 aa, synthetic construct
MDFQVQIFSFLLISASVILSRGQIVLTQSPAIMSASPGEKVTMTCSASSSVSYMYWFQQKPGSSPRLLIYDTSNLASGVPVRFSGSGSGTSYSLTISRMEAEDAATFYCQQWSSYPLTFGAGTKLELK

SEQ ID NO: 51 - DNA, 402 bp, synthetic construct
  source 1..402: mol_type = other DNA
atgaacttagggctcagcttcattttccttgcccttattttaaaaggtgtccagtgtgaggtgcaactggtggagtctgggggaggcttagtgcagcctggagggtccctgaaactctcctgtgcagcctctggattcactttcagtagctatggcatgtcttgggttcgccagagtccagacaagaggctggaatgggtcgcaaccattaataataatggtggtagcacctattatccagacagtgtgaagggccgattcaccatctccagagacaatgccaagaacaccctgtacctgcaaatgagcagtctgaagtctgaggacacagccgtgtattactgtgcaagagatgcggcctttgactgctggggccaaggcaccactctcacagtctcctca

SEQ ID NO: 52 - DNA, 396 bp, synthetic construct
  source 1..396: mol_type = other DNA
atggattcacaggcccaggttcttatattgctgctgctatgggtatctggtacctgtggggacattgtgatgtcacagtctccatcctccctggctgtgtcagcaggagagaaggtcactatgagctgcaaatccagtcagagtctgctcaacagtagaacccgaaagaactacttggcttggtaccagcagaaaccagggcagtctcctaaactgctgatctactgggcatccactagggaatctggggtccctgatcgcttcacaggcagtggatctgggacagatttcactctcaccatcagcagtgtgcaggctgaggacctggcagtttattactgcaagcaatcttataatctattcacgttcggctcggggacaaagttggaaataaaa

SEQ ID NO: 53 - DNA, 396 bp, synthetic construct
  source 1..396: mol_type = other DNA
atgggatggagctgtatcatcctcttcttggcagcaacagctacaggtgtccactcccaggtccaactgcagcagtctgggcctgagctggtgaggcctggggcttcagtgaagatgtcctgtaagacttcaggctatcccttcagcacctactggatgcactgggtgaaacagaggcctggacaaggccttgaatggattggcatgattaaaccttccaatagtgaaactagtttaaatcagaaattcaaggacaaggccacattgaatgtagacaaatcctccaatacagcctacatgcagctcaacagcctgacatctgaggactctggagtctattactgtgcaagggcaggggactcctggggccagggcaccactctcacagtctcctca

SEQ ID NO: 54 - DNA, 393 bp, synthetic construct
  source 1..393: mol_type = other DNA
atgagtcctgcccagttcctgattctgttagtgctctggattcgggaaaccaacggtgatgttgtgctgacccagactccactcactttgtcggttaccattggacaaccagcctccatctcttgcaagtcaagtcagagcctcttagaaactgatggacagacatttttgaattggttgttacagcggccaggccagtctccaaagcgcctaatctatctggtgtctaaactggactctggagtccctgacaggttcactggcagtggatcagggacagatttcacactgaaaatcagcagagtggaggctgacgatttgggaatttattattgctggcaaggtacacattttccgctcacgttcggtgctgggaccaagctggagctgaaa

SEQ ID NO: 55 - DNA, 414 bp, synthetic construct
  source 1..414: mol_type = other DNA
atgctgttggggctgaagtgggttttctttgttgttttttatcaaggtgtgcattgtgaggtgcagcttgttgagtctggtggaggattggtgcagcctaaagggtcattgaaactctcatgtgcagcctctgatttcaccttcaatacccactccatgaactgggtccgccaggctccaggaaaggttttggaatggattgctcgcataagaagtcaaagtaataattatgcaacatattatgccgattcagtgaaagacaggttcaccatctccagagatgattcacaaaacatgttctatctgcaaatgaacaacttgaaaactgaggacacagccatgtattactgtgctggggattactacggcccctttgcttactggggccaagggactctggtcgctgtctctgca

SEQ ID NO: 56 - DNA, 393 bp, synthetic construct
  source 1..393: mol_type = other DNA
atgaagttgcctgttaggctgttggtgctgatgttctggattcctgcttccagcagtgatgttctgatgacccaaactccactctccctgcctgtcagtcttggagatcaagcctccatctcttgcagatctggtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtggacgttcggtggaggcaccaagctggaaatcaaa

SEQ ID NO: 57 - DNA, 417 bp, synthetic construct
  source 1..417: mol_type = other DNA
atgaacttcgggctcagcttgattttccttgtccttgttttaaaaggtgtccagtgtgaggtgatactggtggagtctggggggggcttagtgaagcctggagggtccctgagactctcctgtgcagcctctggattcactttcagtagccatcccttgtcttgggttcgccagagtccggacaagaggctggaatgggtcgcaagcattagtagtggtggtaggtacacctactatccagacagtgtgaaggggcgactcaccatctccagagacaatgccaagaacatcctgtatttacacatgaacagtctgaggtctgaggacacggccatgtattactgtgcaagacggggctctacggctacggcctggtttgcttactggggccaagggactctggtcactgtctctgca

SEQ ID NO: 58 - DNA, 393 bp, synthetic construct
  source 1..393: mol_type = other DNA
atggagacagacacaatcctgctatgggtgctgctgctctgggttccaggctccactggtgacattgcgctgacccaatctccagcttctttggctgtgtctctagggcagagggccaccatctcctgcaaggccagccaaagtgttgattatgatggtgatagttatatgagctggtaccaacagaaaccaggacagccacccaaactcctcatctatggtgcatccaatctagaatctgggatcccagccaggtttagtggcagtgggtctgggacagacttcaccctcaacatccatcctgtggaggaggaggatgctgcaacctattactgtcagcaaagtaatgaggatctcccgacgttcggtggaggcaccaagctggaaatcaaa

SEQ ID NO: 59 - DNA, 399 bp, synthetic construct
  source 1..399: mol_type = other DNA
atgaaatgcagctgggtcatcttcttcctgatggcagtggttacaggggtcaattcagaggttcagctgcagcagtctggggctgagcttgtgaggccaggggccttagtcaagttgtcctgcaaagcatctggcttcaacattaaagactactatatgcactgggtgaagcagaggcctgaagagggcctggagtggattggatggattgatcctgagaatggtaatactatatatgacccgaagttccagggcaaggccagtataacagcagacacatcctccaacacagtcaggctgcagctcagcagcctgacatctgaggacactgccgtctattactgtgctagagagaggggcgcttactggggccaagggactctggtcactgtctctgca

SEQ ID NO: 60 - DNA, 384 bp, synthetic construct
  source 1..384: mol_type = other DNA
atggattttcaagtgcagattttcagcttcctgctaatcagtgcctcagtcatactgtccagaggacaaattgttctcacccagtctccagcaatcatgtctgcatctccaggggagaaggtcaccatgacctgcagtgccagctcaagtgtaagttacatgtactggttccagcagaagccaggatcctcccccagactcctgatttatgacacatccaacctggcttctggagtccctgttcgcttcagtggcagtgggtctgggacctcttactctctcacaatcagccgaatggaggctgaagatgctgccactttttactgccagcagtggagtagttacccgctcacgttcggtgctgggaccaagctggagctgaaa